(19) United States
(12) Patent Application Publication
(10) Pub. No.: US 2023/0415383 A1
(43) Pub. Date: Dec. 28, 2023

(54) METHOD AND APPARATUS FOR FABRICATING BRITTLE MICRONEEDLE

(71) Applicant: The Boeing Company, Arlington, VA (US)

(72) Inventor: Robert M. Pricone, Libertyville, IL (US)

(21) Appl. No.: 18/546,015

(22) PCT Filed: Feb. 11, 2022
(86) PCT No.: PCT/US2022/016127

§ 371 Date: Aug. 10, 2023

Related U.S. Application Data
(60) Provisional application No. 63/148,585, filed on Feb. 11, 2021.

(51) Int. Cl. B29C 41/30; A61M 37/00; B29C 41/28

(52) U.S. Cl. CPC B29C 41/30; A61M 37/0015; B29C 41/28; A61M 2037/0023; A61M 2037/0046; A61M 2037/0053; B29L 2031/7544

(57) ABSTRACT

A method for forming a microneedle array, wherein the microneedle array includes a base surface and an elongated body portion terminating in a sharp tip, and is attached to a polymeric backing layer, includes the steps of: charging a first liquid comprising sugar and an active drug to a microneedle mold; drying the first liquid; optionally charging a second liquid comprising sugar to the microneedle mold and drying the second liquid; adhering a polymeric backing layer to the base surface of the microneedles formed in the microneedle mold to form the microneedle array. A powder coating method, and apparatus for conducting the methods and producing the product are included also.

### CROSS-REFERENCE TO RELATED APPLICATION

This application claims the benefit of priority to U.S. provisional application 63/148,585, filed on Feb. 11, 2021, also titled “Method and Apparatus for Fabricating Brittle Microneedle.”

### FIELD

This disclosure relates generally to a patch of brittle microstructured needles with a less brittle backing layer. More specifically, it is directed to a medication or vaccine delivery system utilizing such needle patches.

### BACKGROUND

Compositions and devices for wound closure patches are well known. A wound closure device which comprises a series or array of microstructured needles affixed to a backing device is available. Using two spaced arrays, the arrays may be positioned on opposite sides of the wound needing closure. The microneedles close wounds by effectively anchoring or holding power over a wound at the stratum corneum (outermost layer of epidermis) closing the skin together. These devices can be easily applied and removed with little pain, as no adhesive is used and the microneedles are inserted only to a depth of the upper epidermis that essentially is devoid of the pain-producing nerve endings.

For purposes of this application, the term micro-needle is all inclusive of various shapes and cross-sections of the needle, such as conical, pyramidal, blade, rectangle, or other variations, each ending with an elongated sharper end, referenced as the tip. The tip is the exterior end that is intended to enter the skin.

The density of the microneedles may be predetermined and may vary depending upon the size of the device and the application it is used with. For example, the density may be 1 microneedle/cm² to 1 microneedle/10 cm². The pitch between adjacent microneedles may be from 30 μm to 1 cm, wherein pitch is defined as the distance between needles, center point to center point.

Recently, a microneedle array or patch has been developed to deliver a vaccine or drug with less pain than an injection needle and to increase potency of the vaccine. The array is a fingertip-sized patch of 400 tiny needles that delivers spike protein pieces into the skin, where the immune reaction is strongest. The patch goes on like a bandage and then the needles, which are made entirely of sugar, and the protein pieces simply dissolve into the skin. Due to the small size of the needles, the microneedle patch causes no pain or bleeding. Microneedle arrays are being tested on humans to deliver chemotherapy as a treatment for skin cancer, they also hold strong potential for use in vaccination and other treatments.

The technology is particularly promising for delivering vaccines or antibodies to fight pathogens since abrasions to the skin—even very tiny ones—produce an immediate and powerful response from the immune system. Traditional syringe vaccines that enter muscle tissue do not elicit quite as effective of a response; they require a much larger dose of vaccine than microneedles do to achieve the desired immunity or treatment. Therefore, vaccination and medicinal treatment through microneedle array patches can be significantly more effective and faster than using hypodermic needles.

However, because of the brittleness of the water-soluble sugar or sugar-like materials, the very small configuration of the microneedles in terms of reduced cross section from the base to the sharp tip, and the relative rigidity required to assure stability and anchoring of the device when installed on the skin, it has proven difficult to provide an economic and useful method for producing these devices having sugar-based microneedles in an efficient manner and with a durable characteristic that is resistant to cracking and breaking either during the manufacturing process, storage and transit, or the application process.

### SUMMARY

Disclosed herein is a method of forming a microneedle array, wherein the microneedle array includes a base surface and an elongated body portion terminating in a sharp tip. The microneedle array is attached to a polymeric backing layer. The steps comprise: charging a first liquid comprising sugar and an active drug to a microneedle mold; drying the first liquid; optionally charging a second liquid comprising sugar to the microneedle mold and drying the second liquid; adhering a polymeric backing layer to the base surface of the microneedles formed in the microneedle mold to form the microneedle array.

Disclosed herein is a microneedle array that includes: a plurality of microneedles, the microneedles comprising a base surface and an elongated body portion terminating in a tip. The microneedles comprise a sugar and the microneedles are attached at the base surface to a polymeric backing layer.

### BRIEF DESCRIPTION OF THE DRAWINGS

FIG. 1 is a cross-sectional depiction of a first step in a process of making the micro-needle array.

FIG. 2 is a cross-sectional depiction of a first step in a process of making the micro-needle array.

FIG. 3 is a diagram of an example apparatus for making the micro-needle array disclosed herein.

FIG. 4 is a diagram of another example apparatus for making the microneedle array disclosed herein.

### DETAILED DESCRIPTION

The microarrays disclosed herein have an improved material characteristics and may be used for drug or vaccine delivery, for example, in a readily scalable system for rapid deployment and administration of vaccines. The microarrays can also be used for delivery of other medicines. The microarrays are designed for applying a biocompatible material to skin without cracking or breaking due to brittleness.

The present disclosure provides a product and methods for forming an array with a polymeric base and water-soluble and medically-safe microneedles having the requisite physical properties, including the necessary sharpness to facilitate easy penetration of the skin. Although it is possible that the microneedles disclosed herein could be curved, it is contemplated that they are not curved, further distinguishing them from typical wound closure microneedles. This enhances the ease of application and removal, and also facilitates an easier manufacturing process, where skin closure is not needed. The microneedles may be provided with a drug, such as a vaccine, that is absorbable by the skin of a mammal, such as a human.

The method and apparatus disclosed herein uses a needle forming process to impart the geometry and functionality of the needles with a flexible backing layer. Details of certain embodiments of the needles and materials used therein may be found in U.S. Pat. No. 8,834,423, incorporated by reference herein. See, e.g., FIG. 21 on creating a fillet-portion at the base of the microneedle and a beveled or undercut elongated portion. Alternatively, providing undercuts to the microneedles of the microneedle array can be accomplished through techniques and apparatuses disclosed in U.S. Pat. No. 10,279,533. In an example, the microneedles may also conform to the various dimensions and geometry disclose in U.S. Pat. No. 10,279,533.

Material mentioned therein included carboxymethylcellulose, which is composed of derivatized glucose joined via β-(1,4) glycosidic linkages and has glucose as a catalysis product. Other materials useable for the biocompatible microneedles include carbohydrates such as, maltose, galactose, sucrose, and galactose.

Molding the needles, then micro-machining or milling is disclosed as the method for making these microneedles in U.S. Pat. No. 8,834,423. The CMC can be formed into a hydrogel for molding. The bioactive component, e.g., vaccine or medicament, can be introduced via spin-casting into the mold. The bioactive component can be present in an amount of, e.g., 0.1% to 30% by weight of the hydrogel, such as, 5% to 25%, or 10% to 20%. A ratio of CMC to bioactive component in both the hydrogel and dried product, may be, for example, 1:5 to 4:1, 1:3 to 2:1, or 1:2 to 1.5:1. The same amounts and ratios of the other materials recited can be used instead of CMC.

In an embodiment, a multi-layered process can be used. Here the surface of the production molds can be covered with about 50 μl (for molds with 11 mm diameter) of CMC-hydrogel and spin-casted for about 5 minutes. After the initial CMC-hydrogel layer, another 50 μl CMC-hydrogel can be layered over the mold and centrifuged for about 4 hours. After drying, the microneedle arrays can be separated from the molds.

Another material that can be used for the microneedles is poly(lactic-co-glycolic acid) (PLGA) also known as poly(L-lactide-co-glycolide). PLGA based materials and devices have been disclosed to require relatively high temperature (e.g., 135° C. or higher) and vacuum for fabrication. Because of this, if anything, U.S. Pat. No. 8,834,423 teaches away from its use, mentioning it as useable, but only extolling its limitations in comparison to CMC. A carbohydrate or sugar-based matrix can be formed at lower temperature and may be more useful for incorporation of temperature-sensitive bioactive components. PLGA is also very expensive. However, it has been determined that PLGA, such as, RESOlViER LG 855 S, can be a desirable option when combined with the low-cost and flexible backing layer disclosed herein. RESOlViER LG 855 S is an amorphous copolymer of L-lactide and glycolide with an inherent viscosity range of 2.5-3.5 dL/g and a Tg of 58° C. The PLGA, which is used in the needles is a very small component compared to the backing layer, making it a viable option with the multi-material system disclosed herein. In addition, the process disclosed herein does not require a vacuum.

Through research and development, it was determined that an all hard-sugar patch is not practical, it is too brittle to handle and attach to the contours of the body on the skin surface. To resolve this problem, in an embodiment disclosed herein, just the needles are made of hard sugar, but they are bonded to a flexible medical grade plastic film such as DR-100 or PMMA (poly-methyl-methacrylate). The bonding layer could be a medical grade pressure-sensitive adhesive (PSA) such as those used to adhere other patches to the body, e.g., acrylate-based biocompatible adhesives, silicone-based adhesives, or synthetic rubber adhesives.

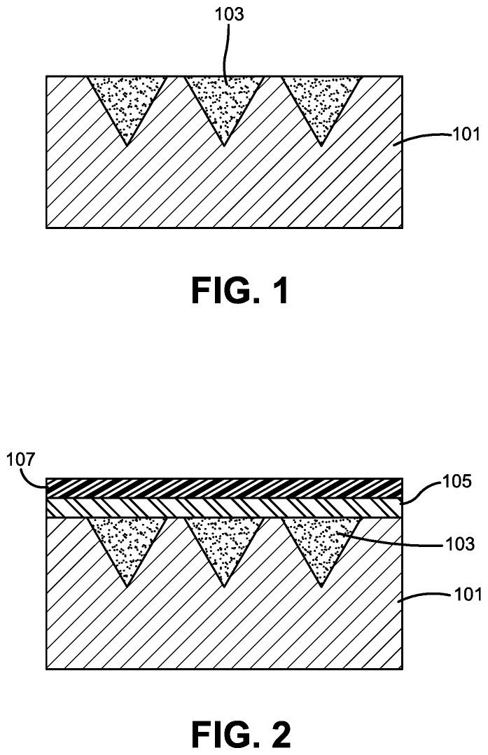

An apparatus and process for constructing an embodiment of a durable, layered microneedle array is disclosed in FIGS. 1-3.

With respect to these figures, a microneedle mold 101 is provided in the form of a long continuous belt. A fluid mixture of microneedle material (e.g., sugar) and drug (e.g. vaccine) is used to fill a first portion (e.g. 20 to 80%, 30 to 70, or 45 to 55%) of the microneedle mold cavity 103 (percentages are based on height of the microneedle or mold depth, 100% being the entire length of the elongated body portion of the microneedle). This may be the tip segment referred to elsewhere herein with respect to heating the microneedles. Alternatively, the sugar and vaccine may be provided as coatings on the first portion of the mold.

The vaccine may be, for example, a vaccine for SARS-Cov-2 (Covid 19). Drugs other than vaccine can be used with this delivery method as well. The drug should be water-soluble and absorbable by the skin and included in an effective amount.

After charging the sugar and bioactive component to the first portion of the mold, the first portion is dried, e.g., by passing the belt through an oven at sufficient temperature and time to fully solidify the needles. Once solidified, a second microneedle layer without the active drug is added to a second portion of the microneedle mold cavity 103, substantially filling the microneedle mold cavity 103, e.g. 90 to 100%, or 95 to 99% of the entire volume of the microneedle mold cavity 103. A second similar drying step is then conducted. This two-step process reduces the cost and may enhance properties of the micro-needle at its base, by putting all the vaccine or drug nearer the tip end of the microneedle (first portion).

After the first portion is charged, or the first and second portion are charged to the microneedle mold, the polymeric backing layer is coupled to or adhered to a base surface of the first or second portion. The base surface is the surface facing the open exterior of the microneedle mold. In an embodiment, the base surface is recessed slightly into the mold, and in an embodiment, the base surface is level with the top of the mold, or even raised slightly above the top of the mold.

In an embodiment, with the microneedle mold cavities 103 filled and dried, a bonding layer 105 is applied in-line. Then the bonding layer 105 is laminated to a flexible acrylic film 107, e.g. a medical grade PMMA to form the brittle, water-soluble sugar microneedle array with a flexible backing layer.

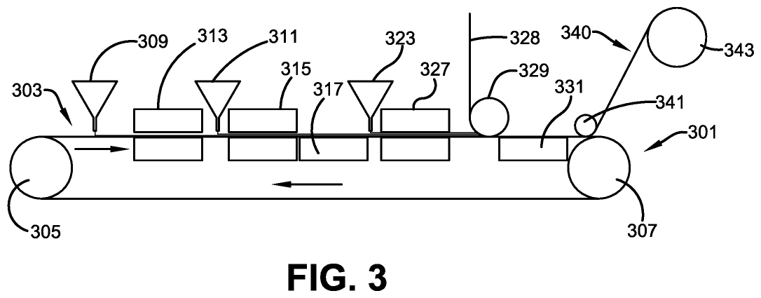

FIG. 3 is a diagram of a continuous manufacturing platform 301 for making the layered microneedle array with a polymeric backing layer and two-layered microneedle with an adhering layer joining them.

The belt 303 is flexible and wrapped around at least two rollers 305, 307, which drive the belt in the clockwise direction. The belt 303 includes the microneedle molds. A first coater 309 is shown at the left on top of the belt mold. This provides the liquefied biocompatible material and drug mixture (coating) into the microneedle molds. The belt 303 proceeds to the right (clockwise) and a second coater 311 is provided for the second charge of biocompatible material. Optionally only one coater may be used to fill the entire or substantially the entire microneedle mold with the mixed biocompatible material and active drug.

After passing the second coater 311 the molds are then filled with the microneedle arrays 321. First and second heater sections 313, 315 are included after the first and second coaters 309, 311 to heat/dry the microneedles in the molds as discussed above. A cooling zone 317 is provided after the second drying/heating zone 315.

In an embodiment, a third coater 323 for an adherent is stationed after the cooling zone 317, and a roll of polymeric backing film 328 may be applied thereafter via, for example, a laminating process, e.g., see the roller 329 with the polymeric backing film 328 on FIG. 3. Alternatively, the film 328 can be pre-coated with the adhesive and applied to the microneedles e.g., by laminating with a roller 329. A final cooling zone 331 is placed near the end of the top part of the track of belt 303. Once all layers are assembled, the polymeric backing film 328 adhered to the microneedles can be pulled off and out of the mold by rollers 341 and 343 as a single sheet having the microneedles as a first water-soluble material (albeit in multiple layers, at least one layer including the active drug) and the backing layer as a second non-water-soluble material. The sheet can then be and cut into smaller dried sheets, e.g., into individual drug delivery strips. These individual strips may be 1 to 10 in², such as 2 to 8 in², or 3 to 7 in².

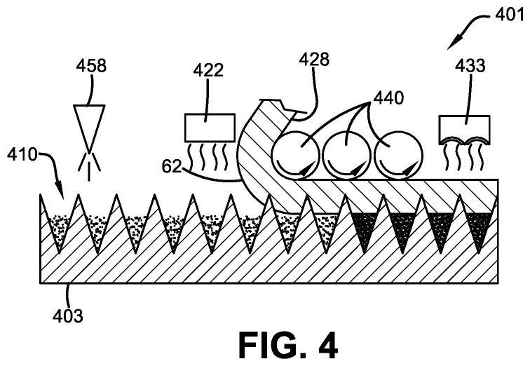

In an embodiment, a specialized powder coating process is used for applying a powdered form of the biocompatible material into the molds and laminating the backing layer directly to the microneedles. FIG. 4 shows a view of a system 401 for powder coating that can be employed in a continuous process as a variation on the FIG. 3 depiction of the continuous system. The process differs from that of FIG. 3, in that a powder coater 458 deposits powder into the molds 410 of the belt 403. The powder can comprise the biocompatible polymer and the vaccine or drug. An electrostatic powder coating technique and apparatus can be used for this process, which includes electrostatically charging the molds 410 to attract the powder from the powder coater 458. In an embodiment, the powder coater can be powered with an electrostatic gun. After depositing the powder, the powder is heated to melting in the molds with a heater 422, which can be an infrared heater. The polymeric backing film 428 is then applied onto the melted top surface of the elongated bodies of the microneedles. Further heating can be applied with a second heating element 433 to improve the thermal bonding between the backing layer and the microneedles. Rollers 440 apply light pressure to the backing layer film as it proceeds along the processing pathway. This apparatus is particularly suitable for applying PLGA powder with a PCL backing layer. It was determined that an adhesive layer can be avoided in this embodiment, as the PLGA and PCL thermally bond to each other. U.S. patent publication 2008/0251964, incorporated herein by reference, can be referenced for further details on this process.

In either process, cooling rolls can be used to further control conditions for drying and solidifying the materials.

The belt 303 including the molds can be long in length, such as 10 to 40 m in length, e.g., 15 to 25 m, or about 20 m long. Width, may be, for example, 1 to 5 m wide, such as 1.5 to 2.5 m, or 2 m. The belt 303 and molds may be made, for example, of silicone, e.g., two-part medical grade silicone, or metal.

In an embodiment, the microneedles have height no greater than 3 mm. In an embodiment, the microneedle includes a base segment and a tip segment. The base segment and tip segment may be differentiated by different material makeup, with the tip segment comprising the biocompatible material and the vaccine or drug (first portion charged), and the base segment including only the biocompatible material (second portion charged). The base surface of the base segment of the microneedle is adhered to the backing layer.

The polymer comprising the backing layer may have an elastic modulus that both permits bending of the array around a skin covered body surface and provides a backing of sufficient stiffness to be able to push the microneedles into the skin surface. Elastic modulus may be determined by ASTM E2769-15. The backing layer may be, for example, 125 μm thick and have an elastic modulus of 2.4 to 1.6 GPa, such 1.86 GPa plus or minus 10%. The elastic modulus of the backing layer is lower than the elastic modulus of the elongated body portion of the microneedles. In an embodiment, the backing layer may range from 25 μm and 250 μm, such as, for example, 75 μm to 200 μm, or 100 μm to 150 μm. The backing layer may be polycaprolactone, PCL.

Another measure of elasticity of the backing layer is dynamic modulus, which may be suitably, 0.3 to 5 N/% stretch, such as 0.5 to 3 N/% stretch, or 0.7 to 1.9 N/% stretch. Dynamic modulus can be measured according to Partsch H., et al. “Classification of compression bandages: Practical aspects.” Dermatol. Surg. 2008; 34:600-609.

In an embodiment, the making and removal of the microneedle array can be incorporated into the continuous process, for example, after the backing layer is laminated or otherwise applied to the needles, and the needles are removed from the molds.

In an embodiment, the PLGA polymer is used as the biocompatible material for mixing with the vaccine or drug in the microneedle molds. In this embodiment, the PLGA can be applied as a powder or a particle to fill the microneedle cavities in the mold. This can be done on a batch process, or a continuous process, with the apparatus disclosed in FIG. 3 or similar variation. In the continuous process a pre-measured dusting of powder may be used instead of a pre-liquefied coating. However, once deposited, the material can be heated to flow into the mold. As mentioned above, the needles are a very small percentage of the total patch weight (e.g., 20% to 2%, 15% to 5%, or 12% to 7%) and the backing will be a lower cost polymer, such as PCL (polycaprolactone), which is very flexible, and thermally bonds to the needles to create a flexible patch that can be applied to skin with very hard PLGA needles. The backing layer can be for example, 98% to 80% by weight of the total patch (microneedle plus backing layer), such as 95%, 85%, or 93% to 88%.

In an embodiment, the adherent layer is unnecessary with the PLGA polymer which can be heated to thermally bond directly to the backing layer, e.g., through a lamination process.

In each of the needle arrays, there may be as few as 12 microneedles in an array, spaced about 3 mm from each other in array on the order of 15 mm×15 mm. The array of this size may have as many as 36 needles and spaced 2 mm apart. In an embodiment, the microneedles may have a density on the needle arrays of 0.053 needles/mm² to 0.16 needles/mm², such as 0.08 to 0.14 needles/mm² or 0.1 to 0.12 needles/mm².

The vaccine or medicine is a bioactive component and, in an embodiment, can include at least two different bioactive components. The bioactive component can include an antigen and an adjuvant for a vaccine application. In some embodiments, the bioactive components can comprise dissoluble materials or insoluble (with respect to water) but dispersible materials. The bioactive components can be natural or formulated macro, micro, and/or nano particulates. The bioactive components can also comprise mixtures of two or more of dissoluble, dispersible insoluble materials and natural and/or formulated macro, micro and nano particulates. In an embodiment, the bioactive component is a SARS-COV-2 vaccine, of the MRNA type or traditional deactivated virus type.

What has been described above includes examples of one or more embodiments. It is, of course, not possible to describe every conceivable modification and alteration of the above devices or methodologies for purposes of describing the aforementioned aspects, but one of ordinary skill in the art can recognize that many further modifications and permutations of various aspects are possible. Accordingly, the described aspects are intended to embrace all such alterations, modifications, and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim. The term “consisting essentially” as used herein means the specified materials or steps and those that do not materially affect the basic and novel characteristics of the material or method. Unless the context indicates otherwise, all percentages and averages are by weight. If not specified above, the properties mentioned herein may be determined by applicable ASTM standards, or if an ASTM standard does not exist for the property, the most commonly used standard known by those of skill in the art may be used. The articles “a,” “an,” and “the,” should be interpreted to mean “one or more” unless the context indicates the contrary.

What is claimed is:

1. A method of forming a microneedle array, wherein the microneedle array includes an elongated body portion terminating in a sharp tip, the elongated body portion coupled to a polymeric backing layer, the method comprising the steps of:
charging a first portion of a biocompatible material to a microneedle mold;
drying the first portion;
optionally, charging a second portion of the biocompatible material to the microneedle mold and drying the second portion;
adhering a polymeric backing layer to a base surface of the first or second portion to form the microneedle array;
wherein an elastic modulus of the polymeric backing layer is lower than an elastic modulus of the elongated body portion.

2. The method of claim 1, further comprising charging a bioactive component to the microneedle mold.

3. The method of claim 1, wherein the microneedle mold is on a belt.

4. The method of claim 3, wherein the microneedle array is removed from the microneedle mold, and the charging, drying, and adhering steps are repeated in a continuous process.

5. The method of claim 1, wherein the elongated body portion of the microneedle has a height no greater than 3 mm and is attached to a base segment having a thickness in the range of between 25 μm and 250 μm.

6. The method of claim 1, wherein said microneedles of the microneedle array are in an array having a density of 1 microneedle per cm² to 1 microneedle per 10 cm² and a pitch of 30 μm to 1 cm.

7. The method of claim 1, wherein said elongated body portion has a shape selected from the group consisting of: a pyramid, rectangle, cone, or blade.

8. The method of claim 1, wherein heat for the drying step is applied in a range of 95° C. to 130° C.

9. The method of claim 1, wherein the backing layer comprises a polymer having an elastic modulus of 2.4 to 1.6 GPa.

10. The method of claim 1, wherein the biocompatible material comprises a sugar.

11. The method of claim 1, wherein the step of charging the second portion of the biocompatible material to the microneedle mold and drying the second portion is performed.

12. A microneedle array comprising:
a plurality of microneedles;
the microneedles comprising an elongated body portion terminating in a tip;
the microneedles comprising a biocompatible material; and
the microneedles coupled to a polymeric backing layer;
the polymeric backing layer having an elastic modulus that is lower than the elastic modulus of the elongated body portion of the microneedles.

13. The microneedle array of claim 12, wherein the elongated body portion comprises a first portion including the tip that comprises a sugar and an active drug, and a second portion comprising a sugar that includes a base surface that is coupled to the polymeric backing layer.

14. The microneedle array of claim 12, wherein the biocompatible material is a sugar or a poly(lactic-co-glycolic acid).

15. The microneedle array of claim 12, wherein the elongated body portion comprises a bioactive component, wherein the bioactive component is a vaccine.

16. The microneedle array of claim 12, wherein the backing layer comprises a polymer having an elastic modulus of 2.4 to 1.6 GPa.

17. The microneedle array of claim 12, wherein the backing layer comprises polycaprolactone.

18. A method of forming a microneedle array, wherein the microneedle array includes an elongated body portion terminating in a sharp tip, the elongated body portion coupled to a polymeric backing layer, the method comprising the steps of:
powder coating a powdered biocompatible material to a microneedle mold;
heating the biocompatible material;
coupling a polymeric backing layer directly to a base surface of the heated mixture;
cooling the heated mixture and back layer to form the microneedle array;
wherein an elastic modulus of the polymeric backing layer is lower than an elastic modulus of the elongated body portion of the microneedles.

19. The method of claim 18, wherein no adhesive layer is applied.

20. The method of claim 18, wherein the powdered biocompatible material is poly(lactic-co-glycolic acid).
